(19) United States
(12) Patent Application Publication
(10) Pub. No.: US 2012/0164715 A1
(43) Pub. Date: Jun. 28, 2012

(54) DEVICE FOR PORCESSING A CYTOLOGICAL OR HISTOLOGICAL PREPARATION

(75) Inventor: François De Trazegnies D'ittre, Villejuif (FR)

(73) Assignee: Assistance Publique-Hôpitaux de Paris, Paris (FR)

(21) Appl. No.: 13/389,666

(22) PCT Filed: Aug. 10, 2010
(86) PCT No.: PCT/EP10/61651

§ 371 Date: Mar. 15, 2012

(30) Foreign Application Priority Data
Aug. 10, 2009 (FR) 0955605

(51) Int. Cl. C12M 1/00

(52) U.S. Cl. 435/283.1

(57) ABSTRACT

A device for processing a cytological or histological preparation includes: a carousel, including at least one space for receiving a slide for receiving a cytological or histological preparation, the carousel being rotatably mounted about an axis of rotation; a nebulizing head that passes above the carousel, including the piezoelectric element and a space for the reagent such that the nebulising head expels at least one drop of reagent as a result of the deformation of the piezoelectric element.

The present invention relates to a device for processing a cytological or histological preparation.

A cytological examination makes it possible in particular to discover abnormal, cancerous or pre-cancerous cells contained in samples of various origin: cervix, uterus, sputum, bronchial and alveolar wash, bronchial aspiration, organ puncture, urine, or pleural or peritoneal liquid. The most common cytological examination is the cervical smear.

As is known, cytological examinations consist of removing, on the patient or in a cavity thereof, a sample containing cells, such a sampling being taken for example at the cervix, then carrying out one or more steps with the ultimate aim of obtaining a preparation containing only the cells having to be analysed. This cytological preparation must then be deposited on a glass slide so as to form a concentration of cells obtained by smear, imprint, cytospin or thin film, said slide then being treated by reagents to obtain a microscopic preparation which can be read by an anatomo-cytopathologist.

Histology is the branch of biology involving the study of tissues, mid-way between cytology and anatomy. It aims to explore the composition, structure and renewal of tissues as well as cellular changes therein.

The techniques of analysis by staining mentioned above in the case of cytological samples are also used in the analysis of histological samples.

A first known staining method for techniques of analysis of cytological or histological samples consists in manually performing a specific number of procedures. The staining is carried out with the aid of a tank containing a reagent. The slides on which the biological matter to be examined has been deposited are placed in a magazine. The magazine is then soaked with the reagent in the tank. The reagent covers the biological matter present on the slides, and some parts of said biological matter are stained by affinity, or chemical or physical reaction. After the staining process, which results from the contact between the reagent and the sample, the slide is rinsed at least once so as to remove the excess reagent, then dried before being analysed under a microscope. In the case of hemalum, some of the hemalum will be fixed by affinity to the nucleus of a cell, the rinsing thus making it possible to remove the hemalum present around the nucleus. Rinsing operations, rest periods and sometimes other chemical or physical reactions are generally necessary between each step of contacting of the sample with a coloured reagent. The advantage of this system is that the tank may contain a plurality of magazines immersed in the same reagent at the same time.

This type of manual approach has a number of disadvantages however.

A first disadvantage lies in the risk of contamination of the reagents. For example, cells are lost from a badly fixed preparation, fall into the tank of reagent and thus contaminate the next slides. This contamination then falsifies all the analyses, thus resulting in the reagents being used prematurely and a significant loss of time.

Furthermore, implementation of these manual analysis methods takes quite a long time, and errors linked to handling of the magazines are possible.

A known solution to the above-mentioned problems consists in using staining automatons. Such a solution makes it possible to automatically place magazines containing slides with the biological matter in the reagent tanks. A robotic arm moves the slide magazine precisely in the staining automaton in accordance with a predefined protocol. The simultaneous processing of a plurality of magazines and different programs is then possible. The automaton may also comprise means for drying and heating the slides. The staining automaton thus makes it possible to obtain quicker staining and to reduce the risk of errors.

Such a solution also has some disadvantages, however.

The use of tanks leads to the presence of a large quantity of reagent, although a small quantity of this same reagent will effectively be used for the staining, this overconsumption of reagent leading to an additional cost which is not insignificant.

These substantial volumes of reagent are also likely to result in undesirable polluting waste.

Furthermore, a histological microtome section typically has a thickness of 3 to 6 μm. With microscopic preparation, viewed from above, such a section appears substantially planar, whereas it is in fact formed of peaks and fissures.

The use of one-time immersion tanks therefore does not necessarily make it possible to contact all the parts of the relief when it is first passed through the tank. Although reliefs are less significant in cytology, in particular for simple liquids (blood or cerebrospinal fluid CSF for example), the presence of fibrin or mucus may also induce reliefs for other types of liquid. The reagent and the slide are charged with static electricity. The opposed charges attract and repel, which makes it more difficult for the reagent to gain access to the fissures of the section. A system for stirring the slide in the tank thus allows the reagent to contact the entire section. This procedure is rather long depending on the reagents used, and takes approximately a few minutes per slide.

Based on this, the object of the present invention is to provide a quick and effective device for processing a cytological or histological preparation, said device making it possible to considerably reduce the amount of reagent used and the amount of polluting waste produced, to ensure a decrease in the risk of errors and contamination, and to obtain an improved quality of penetration within the samples by depositing the reagent directly, in particular in the fissures in the section. In addition, this thus cancels out the electrostatic charges of the section and of the reagent.

To this end, the invention proposes a device for processing a cytological or histological preparation, comprising:
a carousel comprising at least one space for receiving a slide for receiving a cytological or histological preparation, said carousel being rotatably mounted about an axis of rotation;
a nebulising head which passes above said carousel, comprising said piezoelectric element and a volume of said reagent such that said nebulising head expels at least one drop of reagent as a result of the deformation of said piezoelectric element.

The device according to the invention may also have one or more of the features below, considered individually or in any technically feasible combination:
said volume of said reagent is integrated in a cartridge;
the device according to the invention comprises a plurality of cartridges which can contain different reagents;
the device according to the invention comprises:
   rinsing means for rinsing the slide passing near said rinsing means;
   drying means for drying the slide passing near said rinsing means;
the rinsing means comprising means for selecting rinsing products and/or reagents which cannot be nebulised;
the device according to the invention comprises a positioning member for the locating of slides placed on the carousel;
the carousel comprises heating means positioned below said at least one space for the slide;
The device according to the invention comprises:
   identification means for identifying each slide, each slide being equipped with an identifier;
   means for obtaining a traceability of the reagent(s) to which each of the slides has been exposed.
The cartridge comprises an anti-overflow system for preventing the blocking of the nozzles of the nebulising head by crystals of reagents;
A coding chip is associated with a cartridge, said coding chip being able to emit traceability information concerning said cartridge.
The nebulising head comprises an automatic cleaning system; and
the nebulising head comprises nozzles having an orifice diameter and a height adapted according to the viscosity of the reagent used.

The invention and its various applications will be better understood after reading the following description and studying the accompanying figures.

The figures are given merely by way of indication and in no way limit the invention. In the figures:

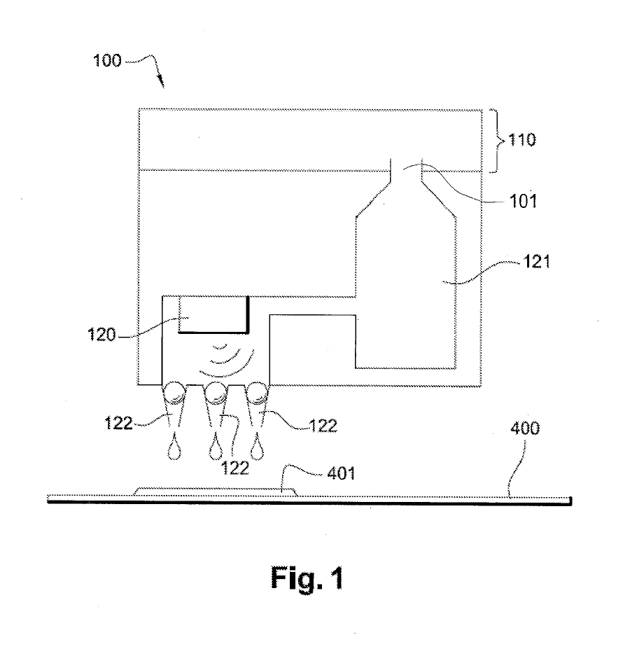

FIG. 1 shows a basic schematic view of a nebulising head with a piezoelectric element of the device according to the invention;

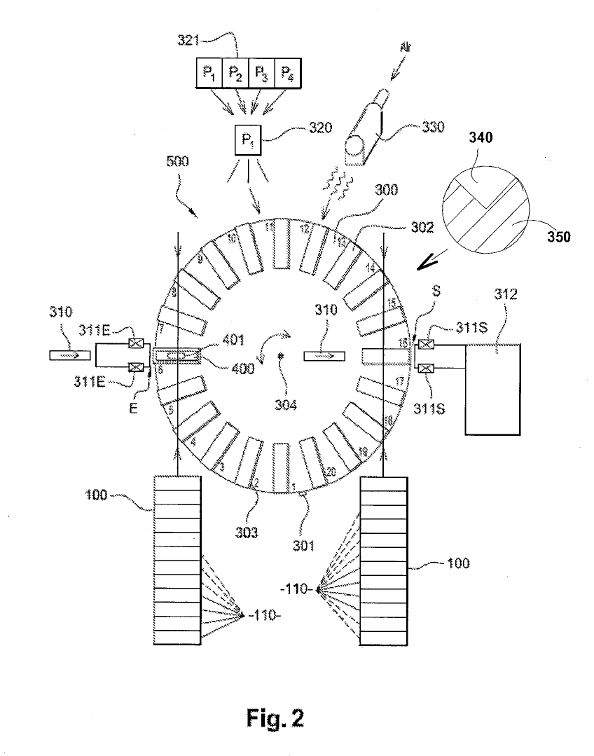

FIG. 2 shows a schematic view of an example of the device according to the invention;

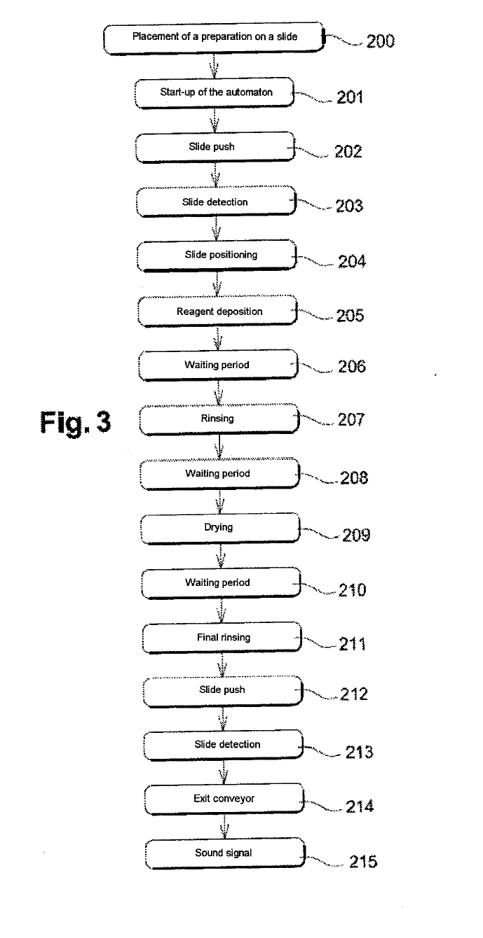

FIG. 3 shows the different steps of the method implemented by the device according to the invention;

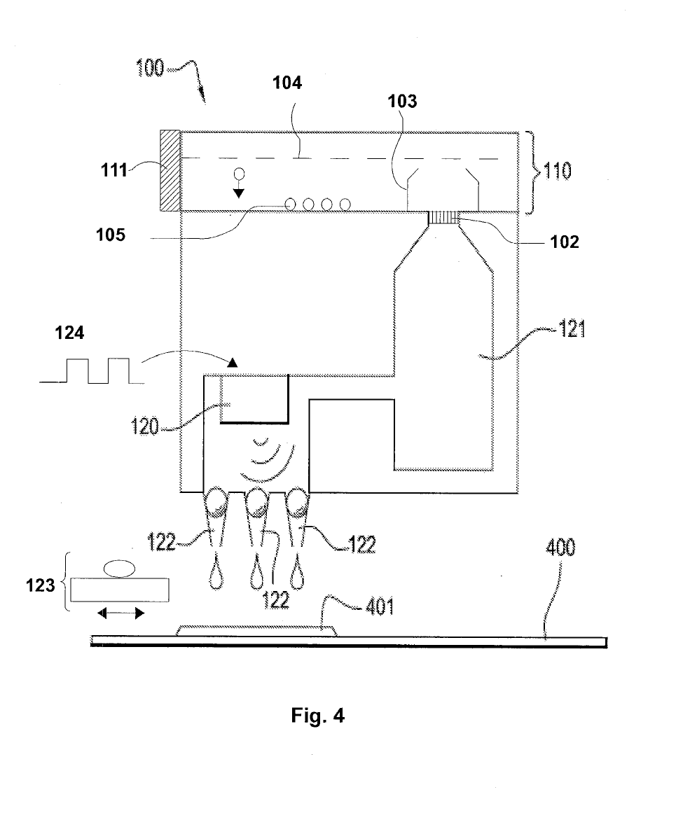

FIG. 4 shows a detailed schematic view of an example of a cartridge and of a nebulising head of the device according to the invention.

Like elements are denoted by like reference signs in all the figures.

In the description below, preparation is understood to mean both cytological preparations and histological preparations, which include for example:
both tissue sections (fixed or fresh);
and spreads (smears or cell spots from a liquid obtained by puncture, cytopuncture or natural discharge).

FIG. 1 shows a basic diagram of a nebulising head 100 with a piezoelectric element 120 of the device according to the invention.

The nebulising head 100 comprises the following elements:
a cartridge 110, containing a reagent;
a vat 121, allowing the arrival of the reagent originating from the cartridge 110;
a piezoelectric element 120, such as a piezoelectric crystal;
a plurality of nozzles (also called injectors) 122 (in this case three nozzles are shown, but at least one nozzle is suffice to implement the described method).

A slide 400 on which a preparation 401 is deposited is located beneath the nozzles 122.

The cartridge 110 contains a reagent for provoking a reaction such as staining when it comes into contact with the preparation 401.

In a non-limiting embodiment, the reservoir of the cartridge 110 is made of a material resistant to the aggressiveness of the reagents contained in said cartridge 110. Each nebulising head 100 comprises a single cartridge 110.

The vat 121 makes it possible to guide the reagent originating from the cartridge 110 to the nozzles 122.

The piezoelectric crystal 120 is electrically charged by an electric field applied by electrical control means (not shown) and is then deflected. This deflection creates a pressure which leads to the ejection of the reagent via the nozzles 122.

The nozzles 122 form perfectly round drops of reagent which are then expelled as a result of the deformation of the piezoelectric crystal 120 when said crystal is exposed to an electric field. It will be noted that the size of the drops is fixed according to the intensity of the electric field applied.

This cartridge 110 contains a type of reagent, said cartridge 110 being disposable. In another embodiment, the reagents can be contained in auxiliary reservoirs enabling automatic refilling of the cartridges without them having to be handled.

FIG. 2 is a schematic view of an example of a device 500 for cytological or histological processing according to the invention.

The device 500 is an automaton comprising the following elements:
a carousel 300 comprising an axis of rotation 304;
a plurality of spaces 302 for receiving a slide 400 (at least one space is necessary);
a positioning member 301;
a slide push 310;
entry sensors 311E and exit sensors 311S;
a conveyor 312;
rinsing means 320 using rinsing products 321 and/or reagents which cannot be nebulised;
drying means 330;
a nebulising head 100, such as that described with reference to FIG. 1, for each reagent.

The slide 400 on which the preparation 401 has been deposited is inserted into a magazine (not shown). The magazine containing at least one slide 400 is then placed at the entry E to the device 500. The slide push 310 pushes the slide 400 selected in the magazine towards the entry sensors 311E. The entry sensors 311E act as a guide to accompany the slide 400 to the space 302 in the carousel 300. The space 302 is identified by a number (from 1 to 20) in relation to the positioning member 301, making it possible to locate the slides 400 on the carousel 300 (the slide is placed in the space 302 at position 6 in this instance). Moreover, each slide 400 is identified by a unique number or barcode. The identifier makes it possible for example to know the type of sample 401 stuck on the slide 400, the name of the patient, and the type of examination to which the sample 401 is subjected. In this case, the automaton 500 comprises identification means making it possible to obtain a traceability of the reagents to which the slide 400 has been exposed during the analysis as well as information relating to the patient. These means are typically software means run by a computer.

When the slide 400 is positioned in its space 302, the nebulising head 100 comprising the cartridge 110 and the selected reagent passes above the slide 400, and by electrical excitation actuating the displacement of the piezoelectric crystal 120, deposits at least one drop of reagent on the preparation 401. To do this, the head 100 moves in translation above the carousel 300.

The carousel 300 turns in a clockwise or anti-clockwise direction about the axis of rotation 304. The rotation of the carousel 300 makes it possible to implement a waiting period before a rinsing step (an example of the method comprising such a step 207 will be described with reference to FIG. 3).

The rinsing means 320 comprise means for selecting products 321. The products 321 are rinsing products, such as alcohol at 70, 95 or 100° C., acidified water, or water; or reagents such as lithium carbonate, ammoniated alcohol or an acid solution. The reagents present in the rinsing means 320 are reagents which cannot be nebulised (that is to say they comprise solid elements likely to block the nozzles of the nebulising head). A tank 340 for recovery and evacuation of excesses of reagents and rinsing products 321 is located below the space for the slides in line with the rinsing means 320 and heating means 330, and more specifically below the slides which are in positions 9 to 12 (as illustrated in FIG. 2).

The products 321 are aspirated by a volumetric pump (four pumps P1 to P4 are shown in this case) before being deposited on the preparation 401. So as to prevent the products 321 from mixing and creating a chemical reaction which could result in the blockage of the openings in the volumetric pump, the rinsing means 320 comprise a separate pump for each one of the products 321. The volumetric pump contains between 5 and 10 millilitres of rinsing product and approximately 2 millilitres for a reagent. The slide 400 is rinsed without pressure so as not to unstick the preparation 401.

A new rotation of the carousel 300 makes it possible to place the slide 400 near the drying means 330. The drying means 330 are an air-drying means for example. Moreover, in the enclosure of the device 500 it is useful, for the preparations 401, to maintain a rather high level of humidity, in particular between 60% and 70%. A water tank 350 is thus installed to regulate the hygrometry inside the device 500, thus making it possible not to dry out the preparations 401 and to avoid evaporation of the reagents. The water tank can be placed below the space for the slides for example.

In the example illustrated in FIG. 2, the water tank 350 and the recovery and evacuation tank 340 form a single cylindrical tank. This tank is located below the carousel 300.

The carousel 300 turns again so that the slide 400 is placed opposite the exit S of said carousel 300. The slide 400 is pushed by the slide push 310, inserting said slide 400 into the magazine and passing said magazine in front of the exit sensors 311S. The magazine thus exits the automaton 500 via the exit conveyor 312, allowing the user to recover the slide 400.

The enclosure of the automaton 500 comprises a filter (not shown) so as to filter the air contained in the automaton 500 and not disturb the analyses of the preparations 401. The filter is activated carbon in particular. The activated carbon traps active solvents, alcohol and chemical products and thus makes it possible to protect users against odours released by the reagents.

According to another embodiment, the device 500 comprises heating means 303 placed below the spaces 302 for the analysis of certain preparations 401, for example with use of silver nitrate. By heating each slide independently, the processing device 500 can thus perform silver stainings.

FIG. 4 shows a schematic example of a cartridge 110 and a nebulising head 100 of the device 500.

It will be noted that, in FIG. 4 (as in FIG. 1), the dimensions of the cartridge 110 and of the vat part 121 with the piezoelectric element 120 and with the nozzles 122 are purely schematic and do not reflect the actual dimensions of these elements. In practice, the cartridge is larger than the vat/piezoelectric element/nozzles assembly. A cartridge 110 is shown with a level of reagent 104 (illustrated in FIG. 4 by a dashed line in the cartridge 110).

The reagent 104 not used within a certain period creates a dead volume. A dead volume is created by crystallisation of the reagent or by normal precipitation of the reagent. At this moment, in the case of crystallisation, crystals 105 of reagents 104 form on the walls and fall to the bottom of the reservoir of said cartridge 110, and in the case of precipitation a deposit of reagent falls to the bottom of the reservoir of said cartridge 110, which could block the nozzles 122 of the nebulising head 100.

The nozzles 122 also become blocked during prolonged use of the nebulising head or when the device 500 is stopped.

So as to avoid blocking the nozzles 122 of the nebulising head 100, the nebulising head comprises:
an anti-overflow system;
an automatic cleaning system.

In non-limiting embodiments, the anti-overflow system may be:
a crystal barrier 103 contained in the cartridge 110 and located at the entry to a passage 101 between the cartridge 110 and the tank 121. In a non-limiting example, the crystal barrier 103 is cylindrical and has a predefined height, making it possible to prevent crystals 105 from accessing the tank 121 and therefore the nozzles 122. The crystal barrier 103 is formed so as to allow the reagent 104 to pass and to retain the crystals 105. The crystal barrier 103 is made of a material which is a metal resistant to the aggressiveness of the reagent 104; and/or
a filter 102 (illustrated in the vertical hatched zone in FIG. 4) placed in the passage 101 between the cartridge 110 and the tank 121. In non-limiting examples the filter can be:
   metal, comprising quite small orifices thus preventing the entry of crystals 105 into the passage 101.
   formed of rot-proof paper.

It will be noted that the crystal barrier 103 and the filter 102 can be used in combination.

It will be noted that, in a non-limiting embodiment, a filter 102 can also be used in the nozzle 122 itself.

In non-limiting embodiments, the automatic cleaning system may be:
A sponge 123 placed level with the nozzles 122 on a system moving in horizontal translation. The passing of the sponge thus makes it possible for drops of reagent 104 to be absorbed at the inlet of the nozzles 122, unblocking the nozzles 122 of the nebulising head 100. The sponge comprises a plurality of small orifices which enable maximum absorption at the inlets of the nozzles 122.
An “auto-cleaning” programme 124 which acts on the piezoelectric element 120 by:
   sending a negative signal at the end of staining. The negative signal is a signal which allows the piezoelectric element to stop on a falling or low edge. This makes it possible to have the least reagent at the exit of the nozzles, a drop of reagent not being expelled from the nozzle and stopping at this moment at the entry to the nozzle 122; and then
   sending a cleaning signal which makes it possible to expel a large drop of reagent to unblock the nozzle and deposit a large amount of reagent in a dustbin.

It will be noted that, on a rising edge, the piezoelectric element is compressed and the drop of reagent is expelled, whereas on a falling edge the piezoelectric element is decompressed and the drop of reagent is not expelled.

It will be noted that, in a non-limiting embodiment, the cartridges 110 and the nebulising head 100 may be integral. This allows the use of a new nebulising head 100 upon each change of cartridge 110. The nebulising head is thus changed often. There is therefore less risk of blocking of the nozzles.

In a non-limiting embodiment, the nebulising head 100 may comprise an anti-return device which prevents a drop of reagent from returning towards the tank 121 when the piezoelectric element is deformed. This ensures that the drop is expelled via the nozzles 122. In a non-limiting example, the anti-return device may be a valve placed between the vat 121 and the piezoelectric element 120.

In a non-limiting embodiment a coding chip 111 is associated with each cartridge 110. This coding chip makes it possible to emit traceability information concerning said cartridge 110. This information may thus be:
the batch number of the reagent in the cartridge;
the level of reagent in the cartridge;
the type of reagent in the cartridge;
the use-by-date of the reagent, etc.

It will be noted that the reagents 104 have different characteristics, in particular with regard to their viscosity.

The nozzle 122 of a nebulising head is also adapted to the viscosity of the reagent 104. The diameter of the orifice in the nozzle 122 of the nebulising head 100 will therefore be larger or smaller depending on the viscosity of the reagent 104. For example, a nozzle 122 of small diameter will be adapted for less viscous reagents 104, a nozzle 122 of average diameter will be adapted for reagents 104 of average viscosity, and a nozzle 122 of large diameter will be adapted for highly viscous reagents 104.

Moreover, the height between the nebulising head 100 and the preparation 401 is adapted according to the type of reagent 104 used. If too close, there may be risks of lifting of cells on the preparation 401. If too far, dead zones may appear between the deposits of reagents (called spot deposit).

It will be noted that a dead zone is an uncovered space between four deposits of reagents. It will be noted that a second passing of nebulisation with a less pronounced offset of the nebulising head in relation to a first passing can be useful to avoid dead zones.

FIG. 3 shows the different steps of the processing method implemented by the device according to the invention.

The different steps of the method have a specific order according to the techniques used. Purely by way of illustration, an order of steps is described hereinafter so as to better understand the method.

The method for processing a preparation 401 comprises the following steps:
In a step 200, called placement of a preparation on a slide 400, the preparation 401 is deposited on the slide 400, then said slide 400 is placed in a magazine located at the entry E to an automaton such as the device 500 described with reference to FIG. 2;
In a step 201, called start-up of the automaton, the operator starts up the automaton 500;
In a step 202, called slide push, the slide 400, placed in the magazine located at the entry E to the automaton 500, is pushed by the slide push 310 so as to enter said automaton 500;
In a step 203, called slide detection, the sensors 311E placed at the entry E to the automaton 500, after the slide push 310, detect the arrival of the slide 400; moreover, the sensors 311E act as a guide for the slide 400;
In a step 204, called slide positioning, after the detection of the slide 400, said slide 400 is then positioned in a space 302 in the carousel 300;
In a step 205, called reagent deposition, once the slide 400 is in its space 302, a passing of the nebulising head 100 above the slide 400 deposits at least one drop of reagent on the preparation 401 by electrical excitation of the piezoelectric element 120;
In a step 206, called a waiting period, after the deposition of reagent, a waiting period is allowed to pass which is defined previously and is necessary for the establishment of the staining reaction;
In a step 207, called rinsing, the preparation 401 thus stained is then placed in front of the rinsing means 320 comprising means for selecting products 321;
In a step 208, called a waiting period, a waiting period defined previously is to be observed between the two steps 207 and 209 of the method;
In a step 209, called drying, the preparation 401 thus rinsed is then placed in front of the drying means 330, in particular air;
In a step 210, called a waiting period, a waiting period defined previously is to be observed between the two steps 209 and 211 of the method;
In a step 211, called final rinsing, the preparation 401 is placed one last time in front of the rinsing means 320 before being analysed;
In a step 212, called slide push, the slide 400 placed in the space 302 in the carousel 300 is pushed by the slide push 310 so as to exit said space 302;
In a step 213, called slide detection, the sensors 311S placed at the exit S of the automaton 500, after the slide push 310, detect the exit of the slide 400 and guide the slide 400 in the magazine;
In a step 214, called exit conveyor, the slide 400 exiting the automaton 500 is placed on the conveyor 312, making it possible for said slide 400 to exit said automaton 500 entirely;
In a step 215, called sound signal emission, the slide 400 exiting the automaton 500 is then signalled to the operator by a signal such as a sound signal or a visual signal.

Additional steps may supplement the method, for example:
a step of cleaning of the nebulising head 100 makes it possible, at the end of use of the automaton 500, to prevent the residues of reagents from blocking the nozzles 122;
a step of evacuating excesses of reagents and/or rinsing products 321.

The technique called “drop-on-demand” (DOD) by piezoelectrics is thus used to apply the analysis of cytology or histology slides. This nebulisation technique is particularly adapted to this type of cytological or histological analysis, conversely to other nebulisation techniques such as thermal injection, which consists in heating the product to be expelled under pressure and which has two major disadvantages: the need to heat the product to be expelled is incompatible with the reagents used in histological or cytological analyses, and the fact that the size of the drops expelled is fixed. By contrast, the technique of DOD by piezoelectrics does not require any heating and makes it possible to achieve different sizes of drops according to the intensity of the electrical signal sent to the piezoelectric element.

The benefit of nebulising the reagents is to contact all points of a relief during a first passing, which makes it possible to save time compared to section-reagent contact by one-time immersion. The surface tension of the reagents as well as the cell/slide/reagent electrostatic charges are reduced to the maximum, which makes it possible to obtain a particularly effective quality of penetration. The method could even reduce the slowing of the penetration of reagents within cells, this being caused by lipid substances.

This method can be automated very easily in an automaton incorporating rinsing systems, heating systems and/or systems for controlling physical parameters (heat, time), as indicated above.

Lastly, the use of drops of reagents allows a considerable saving of reagents and a significant decrease in any polluting waste.

The automaton is able to analyse slides in the field of standard staining, such as HES (hemalun, eosine, safran) or for more specific implementations. The method can also be used for applications with cryostat sections.

The magazine comprises a plurality of slides, for example slides for an automaton comprising 20 spaces. The automaton comprises detection and selection means making it possible to detect a given slide and to insert it into the automaton.

When a slide has to be analysed urgently, the operator can modify the course of the slides so that the slide to be processed urgently is analysed more quickly.

The cartridges of nebulising heads are monitored so that their level and also the last date on which they were changed are known. The reagents may be monitored by identification means making it possible to obtain a traceability of the reagents to which the slides have been exposed.

According to another embodiment, the automaton may be a linear automaton such that the slide is moved along the automaton to follow the different steps of the method; apart from the carousel, this linear automaton comprises the same means as the automaton 500 described with reference to FIG. 2. However, it could also be formed so as to be more compact than the automaton 500 of FIG. 2. Moreover, a system comprising a plurality of tanks may be placed outside the device 500, making it possible to remove paraffin from the slides 400 comprising a preparation 401 before being exposed to reagent(s). In particular, the system comprises a tank of xylene, a tank of alcohol and a tank of water. The removal of paraffin is useful in particular for the processing of a histological preparation. Since the removal of paraffin is known to a person skilled in the art, it is not described in detail here.

The processing device is thus adapted in particular to cytology and to work on fresh sections supplied by cryostat “extemporaneous examinations”. The processing device is also adapted to immunohistochemistry and to the field of chemistry.

1. A device for processing a cytological or histological preparation, the device comprising:
a carousel comprising at least one space for receiving a slide for receiving a cytological or histological preparation, said carousel being rotatably mounted about an axis of rotation, and
a nebulising head which passes above said carousel, comprising a piezoelectric element and a volume of reagent such that said nebulising head is configured to expel at least one drop of reagent as a result of the deformation of said piezoelectric element.

2. The device according to claim 1, wherein said volume of said reagent is integrated in a cartridge.

3. The device according to claim 2, comprises comprising a plurality of cartridges which are configured to contain different reagents.

4. The device according to claim 1, comprising:
a rinser configured to rinse the slide passing near said rinser;
a dryer configured to dry the slide passing near said rinser.

5. The device according to claim 4, wherein the rinser comprises a selector configured to select and/or reagents which cannot be nebulised.

6. The device according to claim 1, comprising a positioning member for locating slides placed on the carousel.

7. The device according to claim 1, wherein the carousel comprises a heater positioned below said at least one space for the slide.

8. The device according to claim 1, comprising:
an identifier configured to identify each slide, each slide being equipped with an identifier;
a tracer configured to obtain a traceability of the reagent(s) to which each of the slides has been exposed.

9. The device according to claim 2, wherein the cartridge comprises an anti-overflow system configured to prevent the blocking of the nozzles of the nebulising head by crystals of reagents.

10. The device according to claim 2, wherein a coding chip is associated with a cartridge, said coding chip being able to emit traceability information concerning said cartridge.

11. The device according to claim 1, wherein the nebulising head comprises an automatic cleaning system.

12. The device according to claim 1, wherein the nebulising head comprises nozzles having an orifice diameter and a height adapted according to the viscosity of the reagent used.
